(19) United States
(12) Patent Application Publication
(10) Pub. No.: US 2010/0292478 A1
(43) Pub. Date: Nov. 18, 2010

(54) PROCESS OF PREPARING A RADIOACTIVE COMPOUND CONTAINING A FLUORINE-18 ISOTOPE

(75) Inventors: Zang Hee CHO, Incheon (KR); Young Bo Kim, Seongnam-si (KR); Sang Yoon LEE, Incheon (KR); So Hee KIM, Incheon (KR)

Correspondence Address: PATTERSON THUENTE CHRISTENSEN PEDERSEN, P.A., 4800 IDS CENTER, 80 SOUTH 8TH STREET, MINNEAPOLIS, MN, 55402-2100, US

(73) Assignee: GACHON UNIVERSITY OF MEDICINE & SCIENCE INDUSTRY- ACADEMIC COOPERATION FOUNDATION, Incheon (KR)

(21) Appl. No.: 12/749,046

(22) Filed: Mar. 29, 2010

(30) Foreign Application Priority Data
May 18, 2009 (KR) 10-2009-0043292

(51) Int. Cl. C07D 455/06; C07D 451/00; C07D 213/64; C07D 277/62; C07C 253/30; C07C 217/76; C07C 45/61

(52) U.S. Cl. 546/95; 546/132; 546/300; 548/178; 558/401; 564/442; 568/315

(57) ABSTRACT

A process of preparing a radioactive compound containing a fluorine-18 isotope is provided. In one embodiment, the process may comprise forming a [¹⁸F] fluoroalkyl triflate by triflating a [¹⁸F] fluoroalkyl compound with AgOTf, and forming a [¹⁸F] fluoroalkylated radioactive compound through alkylation between the [¹⁸F] fluoroalkyl triflate and a radioactive compound precursor having at least one group selected from NH, OH and SH.

The present application claims priority to Korean Patent Application No. 10-2009-43292, filed on May 18, 2009, the subject matter of which is incorporated herein by reference in its entirety.

### BACKGROUND

The present disclosure generally relates to a process of preparing a radioactive compound and more particularly to a process of preparing a radioactive compound comprising a fluorine-18 isotope.

A radioactive isotope is an isotope that decays to a stable state while emitting radioactive rays. Each radioisotope has a unique half-life, which is the period of time, for the radioisotope undergoing decay, to decrease by half in terms of radioactivity irrelevant to the environment. Positron emission tomography (PET), which is recently attracting a lot of attention in the diagnosis and research of various diseases in the medical field, detects positrons emitted by radioactive isotopes, such as carbon-11 and fluorine-18. Fluorine-18, and carbon-11 emit positrons which immediately react with electrons (water) to be annihilated, resulting in the emission of two photons in opposite directions with an energy of 512 keV. Such spatial characteristics make it possible for PET to produce a three-dimensional (3D) tomography. Radioactive isotopes for PET are typically produced directly where the radioisotopes are to be used by a small size cyclotron with a low energy of 5 to 30 MeV.

Since the half-life of radioisotopes is relatively short, a desired radioactive pharmaceutical is prepared quickly, analyzed for determination of its quality, and then injected into patients or animals. For example, a radiopharmaceutical that serves as a tracer can be prepared by labeling a substance that is identical or similar to metabolic substances that increase under specific disease conditions with the above radioactive isotopes. Such radiopharmaceuticals can also be made by labeling a compound that is identical or similar to a substance that bonds with a specific receptor. Radiopharmaceuticals prepared by such methods are administered into a body, and then the distribution of the isotopes is measured and analyzed to obtain useful data.

The development of highly efficient methods for synthesizing radiopharmaceuticals is very important academically and economically, since the fast production of radiopharmaceuticals having high quality and high yield could enhance the quality of tomography results, enable more patients to be scanned, and allow the pharmaceuticals to be also used at nearby medical facilities.

In fields such as PET where radioactive rays emitted by fluorine-18 are detected, there have been attempts to synthesize radioactive compounds where a [¹⁸F] fluoroalkyl group is attached to the nucleophilic elements of the compound, such as nitrogen, oxygen or sulfur.

For example, References 1, 5 and 6 disclose one-step synthetic processes where a radioactive compound precursor directly reacts with fluorine-18. The radioactive compound precursor used in the above process has an alkyl group with a highly reactive leaving group, where the alkyl group is attached to a nucleophilic element of the precursor (see FIG. 1). The one-step synthetic process may be easily carried out since it comprises only one step, but it has disadvantages such as low radiochemical yield and low specific radioactivity of the final product.

Further, there is a two-step synthetic process where a [¹⁸F] fluoroalkyl compound is prepared by labeling an alkyl compound with fluorine-18, and then attaching the [¹⁸F] fluoroalkyl compound to a nucleophilic element of the radioactive compound precursor (see FIG. 2). Reference 2 discloses a process where 3-bromo-1-[¹⁸F] fluoropropane is prepared from 3-bromopropyl-1-triflate and reacted with a radioactive compound precursor, i.e. nor-β-CIT, to produce [¹⁸F] FP-CIT. Reference 3 discloses a process where 3-bromo-1-[¹⁸F] fluoropropane is prepared from 1,3-dibromopropane and reacted with nor-β-CFT, a radioactive compound precursor, to produce [¹⁸F] β-CFT-FP. Reference 3 also discloses a process where [¹⁸F] fluoropropyltosylate is prepared from TsO—(CH₂)₃—OTs (OTs is a tosylate group) and reacted with nor-β-CFT to produce [¹⁸F] β-CFT-FP. Reference 4 discloses a process where 3-halogenated 1-[¹⁸F] fluoropropane is prepared from an alkyl precursor and reacted with nor-β-CFT to produce [¹⁸F] β-CFT-FP. However, the above two-step synthetic processes may take a long time since they involve two steps and provide a lower radiochemical yield compared to a one-step synthetic process. On the other hand, both substitution reactions and elimination reactions by fluorine-18 take place competitively in the one-step synthetic process, while elimination reactions hardly occur in the two-step synthetic process. Thus, two-step synthetic processes generally provide better specific radioactivity than one-step synthetic processes.

Reference 1: Radiosynthesis of [¹⁸F]N-3-Fluoropropyl-2-β-Carbomethoxy-3-β-(4-Iodophenyl) Nortropane and the First Human Study With Positron Emission Tomography, NMB, 1996

Reference 2: Synthesis of a Dopamine Transporter Imaging Agent, N-(3-[¹⁸F]Fluoropropyl)-2-carbomethoxy-3-(4-iodophenyl)nortropane, Korean J Nuc Med, 1999

Reference 3: Preparation of [¹⁸F] β-CFT-FP and [¹¹C] β-CFT-FP, selective radioligands for visualization of the dopamine transporter using positron emission tomography (PET), JLCR, 2000

Reference 4: Synthesis of N-(3-[¹⁸F]Fluoropropyl)-2β-carbomethoxy-3β-(4-iodophenyl)nortropane ([¹⁸F]FP-β-CIT), JLCR, 2006

Reference 5: A New Class of SN₂ Reactions Catalyzed by Protic Solvents: Facile Fluorination for Isotopic of Diagnostic Molecules, JACS, 2006

Reference 6: One-step high-radiochemical-yield synthesis of [¹⁸F]FP-CIT using a protic solvent system, NMB, 2007

### SUMMARY

The present disclosure provides an improved process for increasing the radiochemical yield of a radioactive product while maintaining the advantages of the conventional two-step synthetic process of providing a high level of specific radioactivity.

In one embodiment by way of non-limiting example, a process of preparing a radioactive compound is provided that comprises: forming a [¹⁸F] fluoroalkyl triflate by triflating [¹⁸F] fluoroalkyl compound with AgOTf (silver triflate or silver trifluoromethanesulfonate), and forming a [¹⁸F] fluoroalkylated radioactive compound by reacting the [¹⁸F] fluoroalkyl triflate with a radioactive compound precursor having at least one group selected from NH, OH and SH.

In another embodiment by way of non-limiting example, a process of preparing a radioactive compound comprises:

forming a compound of Formula 3 as follows

[¹⁸F]F—CₙH₂ₙ—OTf (3)

by reacting a compound of Formula 2 as follows

[¹⁸F]F—CₙH₂ₙ—X (2)

where n is an integer from 2 to 6, and X is any one of Cl, Br and I,
with AgOTf; and

forming a radioactive compound containing a fluorine-18 isotope by reacting the compound of Formula 3 with a radioactive compound precursor having at least one group selected from NH, OH and SH.

In another embodiment by way of non-limiting example, a process of preparing a radioactive compound comprises:

forming a compound of Formula 2 as follows

[¹⁸F]F—CₙH₂ₙ—X (2)

by subjecting a compound of Formula 1 as follows

X′—CₙH₂ₙ—X (1)

where n is an integer from 2 to 6, X′ is any one selected from the group consisting of TsO, NsO, MsO, TfO, BsO, Cl, Br and I, and X is any one of Cl, Br and I,
to substitution with a fluorine-18 isotope;

heating the compound of Formula 2 to its boiling point or above;

forming a compound of Formula 3 as follows

[¹⁸F]F—CₙH₂ₙ—OTf (3)

by reacting the compound of Formula 2 with AgOTf; and

forming the radioactive compound containing a fluorine-18 isotope by reacting the compound of Formula 3 with a radioactive compound precursor having at least one group selected from NH, OH and SH.

The above Summary was provided to introduce selected concepts in a simplified form that are further described below in the Detailed Description. The above Summary is not intended to identify key features or essential features of the claimed subject matter, nor is it intended to be used to limit the scope of the claimed subject matter.

### BRIEF DESCRIPTION OF THE DRAWINGS

FIG. 1 shows an illustrative process for preparing a radioactive compound by the conventional one-step synthetic process.

FIG. 2 shows an illustrative process for preparing a radioactive compound by the conventional two-step synthetic process.

FIG. 3 shows an illustrative embodiment of a process for preparing a radioactive compound according to the present disclosure.

FIG. 4 is a flowchart schematically showing the steps of an illustrative preparation process according to the present disclosure.

FIG. 5 is a diagram illustrating the arrangement of equipments for conducting a consecutive reaction process according to the present disclosure.

FIG. 6 depicts radiochemical yield data for [¹⁸F] FP-CIT measured with a TLC device for radioactivity measurement right after completing an illustrative embodiment of a process according to the present disclosure.

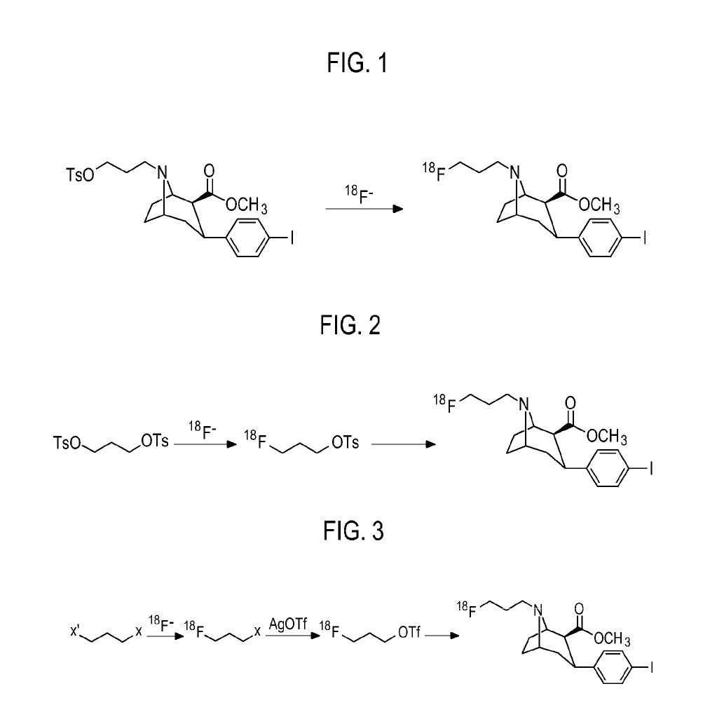

### DETAILED DESCRIPTION

In the following detailed description, reference is made to the accompanying drawings, which form a part hereof. The illustrative embodiments described in the detailed description, drawings, and claims are not meant to be limiting. Other embodiments may be utilized, and other changes may be made, without departing from the spirit or scope of the subject matter presented here. It will be readily understood that the components of the present disclosure, as generally described herein, and illustrated in the Figures, may be arranged, substituted, combined, and designed in a wide variety of different ways, all of which are explicitly contemplated and make part of this disclosure. Those of ordinary skill will appreciate that, for the methods disclosed herein, the functions performed in the methods may be implemented in differing order. Furthermore, the outlined steps are provided only as examples, and some of the steps may be optional, combined into fewer steps, or expanded to include additional steps without detracting from the essence of the present disclosure.

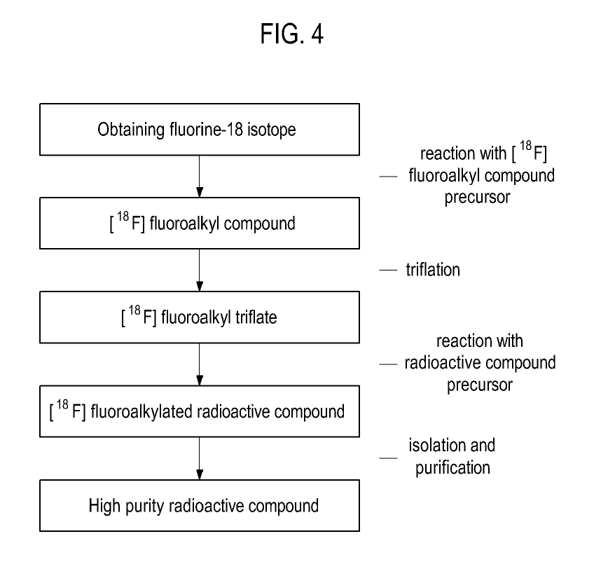

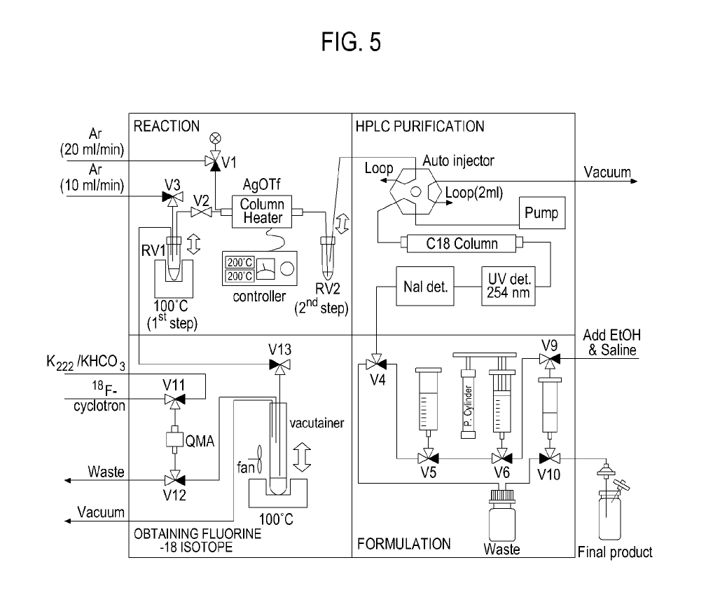

FIG. 4 is a flowchart schematically showing the steps of an illustrative preparation process according to the present disclosure. FIG. 5 is a diagram illustrating the arrangement of equipments for conducting a consecutive reaction process according to the present disclosure. Referring to FIGS. 4 and 5, one embodiment of the present disclosure by way of non-limiting example is outlined as follows:

First, fluorine-18 isotopes are obtained (FIG. 5, lower left part). A [¹⁸F] fluororoalkyl compound is obtained by labeling a [¹⁸F] fluoroalkyl compound precursor with the fluorine-18 isotope. Then, a [¹⁸F] fluoroalkyl triflate is obtained by triflating the [¹⁸F] fluoroalkyl compound. The obtained [¹⁸F] fluoroalkyl triflate is reacted with the radioactive compound precursor to thereby produce a [¹⁸F] fluoroalkylated radioactive compound (FIG. 5, upper left part). A highly pure radioactive compound can be obtained by additionally isolating and purifying the [¹⁸F] fluoroalkylated radioactive compound (FIG. 5, upper right part). Compositions containing the radioactive compound and other substances may be manufactured as desired, for example, in the form of an injection formulation to be administered to humans (FIG. 5, lower right part).

The method of obtaining the fluorine-18 isotopes is described. The fluorine-18 isotopes may be produced by using any methods known in the art including methods using a cyclotron. When using the cyclotron, fluorine-18 isotopes may be obtained in an aqueous solution. The aqueous solution is passed through, e.g., a QMA light cartridge (commercially obtainable from Waters, Inc.) where only the fluorine-18 isotopes are adsorbed onto the cartridge. The adsorbed isotopes are eluted using an acetonitrile solution containing Kryptofix 2.2.2. (K₂₂₂, commercially obtainable from Sigma Aldrich Corp.) and potassium (bi)carbonate or tetra-N-butyl-ammonium (bi)carbonate, and then water and organic solvent are evaporated from the eluted solution to obtain a dried mixture comprising the fluorine-18 isotopes.

The process of obtaining a [¹⁸F] fluoroalkyl compound by reacting the fluorine-18 isotopes with a [¹⁸F] fluoroalkyl compound precursor is described. In the present disclosure, a [¹⁸F] fluoroalkyl compound precursor refers to a compound which can form a [¹⁸F] fluoroalkyl compound by reacting with a fluorine-18 isotope. The [¹⁸F] fluoroalkyl compound precursor has at least one leaving group that can be substituted with fluorine-18. The leaving group may include, but is not limited to, tosylate (TfO), nosylate (NsO), bosylate (BsO), mesylate (MsO), triflate (TfO), Cl, Br, I, SR₂ (R is an alkyl), OH₂, NR₃(R is an alkyl), CH₃COO etc., and more specifically TsO, NsO, BsO, MsO, TfO, Cl, Br and I. The [¹⁸F] fluoroalkyl compound precursor may further comprise a group that can be substituted with TfO in the subsequent step. The group that can be substituted with TfO may be any group known in the art to be suitable for the triflation reaction. For example, the group may include, but is not limited to, Cl, Br and I. The alkyl main chain of the [¹⁸F] fluoroalkyl compound precursor may be a straight, branched or cyclic chain comprising at least one carbon atom.

In one embodiment, the [¹⁸F] fluoroalkyl compound precursor may have the following formula:

X′—CₙH₂ₙ—X (1)

wherein n may be an integer from 1 or more, or an integer from 1 to 10, specifically 2 to 6, more specifically 2 to 4 or even more specifically 2 and 3. The alkyl main chain of Formula 1 may be straight, branched or cyclic, particularly straight or branched, and more particularly straight.

In the above formula, Group X′ represents a leaving group that can be substituted with fluorine-18. The leaving group may include, but is not limited to, tosylate (TfO), nosylate (NsO), bosylate (BsO), mesylate (MsO), triflate (TfO), Cl, Br, I, SR₂ (R is an alkyl), OH₂, NR₃(R is an alkyl), CH₃COO etc. More specifically, the leaving group may be any one selected from the group consisting of TsO, NsO, BsO, MsO, TfO, Cl, Br and I. The leaving group X′ may be attached to any position on the alkyl main chain.

In the above formula, Group X represents a group that can be substituted with TfO in the subsequent step. The group that can be substituted with TfO may be any one known in the art to be suitable for a triflation reaction, while Cl, Br or I may be useful when AgOTf is used as the triflating agent. The group X may be attached to any position on the alkyl main chain.

The reaction for labeling the [¹⁸F] fluoroalkyl compound precursor with fluorine-18 may be carried out under conditions commonly practiced in the art. In one embodiment, the above-mentioned dried mixture containing fluorine-18 is added to a solution comprising the [¹⁸F] fluoroalkyl compound precursor, and the combined solution is heated while stirring.

The process of obtaining [¹⁸F] fluoroalkyl triflate by triflating the [¹⁸F] fluoroalkyl compound is described. In the present disclosure, a [¹⁸F] fluoroalkyl compound refers to a compound that functions as a precursor of the [¹⁸F] fluoroalkyl triflate. As described above, the group that can be substituted with TfO may be selected from a group known to be suitable for triflation reaction, and may be Cl, Br or I, if necessary.

In another embodiment, the [¹⁸F] fluoroalkyl compound may have the following formula:

[¹⁸F]F—CₙH₂ₙ—X (2)

where n is an integer from 1 or more, or from approximately 1 to 10, specifically 2 to 6, more specifically 2 to 4, or even more specifically 2 and 3. The alkyl main chain of Formula 2 may be straight, branched or cyclic, specifically straight or branched, or more specifically straight.

In the above formula, X is a group that can be substituted with TfO in the subsequent step. The group that can be substituted with TfO may be selected from a group known to be suitable for triflation reaction, and Cl, Br or I may be useful when AgOTf is used as the triflating agent. The group X may be attached to any position on the alkyl main chain.

In the present disclosure, the [¹⁸F] fluoroalkyl triflate has a structure where a portion of the [¹⁸F] fluoroalkyl compound is substituted with a TfO group. In one embodiment, [¹⁸F] fluoroalkyl triflate may have the following Formula 3 which corresponds to Formula 2 where X is substituted with TfO.

[¹⁸F]F—CₙH₂ₙ—OTf (3)

where n and the form of the chain are defined as described above in relation to Formula 2.

The reagent used for triflating the [¹⁸F] fluoroalkyl compound may be a known triflate salt, specifically a triflate salt of lithium, sodium, tin, aluminum, copper, erbium, europium, ammonium, barium, calcium, cerium, ruthenium, magnesium, neodymium, potassium, samarium, holmium, indium, terbium, thulium, yttrium, scandium, zinc or silver (commercially obtainable from Sigma Aldrich Corp., GFS Chemicals Inc. or Solchemar Lda, etc.). More specifically, the triflating agent may be AgOTf.

AgOTf may be used in the form of a heated AgOTf column. The column is filled with AgOTf and heated. The column may be quartz or Pyrex material having certain lengths and inside diameters. AgOTf may be mixed with sand, such as sea sand (commercially obtainable from Sigma Aldrich Corp.), to be a homogeneous mixture. The mixture is placed in the column and both ends of the place of the mixture may be blocked with glass wool to prevent the triflating agent from leaking out of the column when gas passes through the column. A column heater made with a hotwire may be installed on the AgOTf column to evenly heat the filled part of the column. The AgOTf column may be heated to a temperature not greater than the melting point of AgOTf (i.e. about 365 to 367° C.).

In the AgOTf column heated to high temperature, the [¹⁸F] fluoroalkyl compound may exist as a gas phase or liquid phase before it reacts with AgOTf. The reason the [¹⁸F] fluoroalkyl compound exists in a gas phase may have been because the compound was introduced into the AgOTf column in a gas state, or because the compound was introduced in a liquid state but vaporized by the high temperature of the AgOTf column. The reason the [¹⁸F] fluoroalkyl compound exists in a liquid phase may have been because the compound was introduced into the column in a liquid state, or the compound was introduced in a gas state but was condensed to liquid due to the lower column temperature compared to the boiling point of the [¹⁸F] fluoroalkyl compound.

In another embodiment of the present disclosure, the compound of Formula 3 may be produced by a process which comprises: forming the compound of Formula 2 by subjecting the compound of Formula 1 to substitution with a fluorine-18 isotope in a reaction container, evaporating the compound of Formula 2 from the container by heating, and forming the compound of Formula 3 by triflating the compound of Formula 2 in the heated AgOTf column.

X′—CₙH₂ₙ—X (1)

[¹⁸F]F—CₙH₂ₙ—X (2)

[¹⁸F]F—CₙH₂ₙ—OTf (3)

where X′ is any one selected from the group consisting of TsO, NsO, MsO, TfO, BsO, Cl, Br and I, X is any one of Cl, Br and I, and n is a integer from 2 to 6, more specifically 2 to 4 or even more specifically 2 and 3.

When the compound of Formula 2 is formed by reacting the compound of Formula 1 with fluorine-18, the un-reacted compound of Formula 1 may be present in the reaction container. If the reaction container is heated, the compound of Formula 2 is vaporized and transformed into gas. Since the compound of Formula 1 has a higher molecular weight than the compound of Formula 2, the compound of Formula 1 is generally not easily evaporated compared to the compound of Formula 2. The heating temperature may be not lower than the boiling point of the compound of Formula 2 and not higher than the boiling point of the compound of Formula 1. Within this temperature range, the compound of Formula 2 actively vaporizes while the compound of Formula 1 does not. The vaporized compound of Formula 2 may be transferred into the AgOTf column in a gas phase. Alternatively, the compound of Formula 2 may be liquidized in the transfer conduit to the AgOTf column but transformed into gas in the heated column. In the heated AgOTf column, the gaseous compound of Formula 2 is transformed into the compound of Formula 3 through a triflation reaction. The formation of impurities is minimized since the un-reacted compound of Formula 1 hardly flows into the heated AgOTf column.

If n is 1, however, there is the possibility of a large amount of un-reacted compound flowing into the heated AgOTf column when the compound of Formula 2 is evaporated from the reaction container by heating. For example, in the consecutive process of producing [¹⁸F] F—CH₂—OTf from Br—CH₂—Br via [¹⁸F] F—CH₂—Br, the un-reacted Br—CH₂—Br may remain in the reaction container even if Br—CH₂—Br is substituted with fluorine-18. If the reaction container is heated to vaporize the reaction product, it is hard to avoid the incorporation of the volatile Br—CH₂—Br into the gas phase. Thus, a large amount of impurities, other than [¹⁸F] F—CH₂—OTf, can be produced in the heated AgOTf column. In order to prevent the incorporation of Br—CH₂—Br into the heated AgOTf column, there is a need to install a filtering system before the AgOT column.

On the other hand, if n is greater than 6, it is difficult to vaporize the compound of Formula 2 since the compound has a high molecular weight and a very high boiling point.

The AgOTf column may be heated to a temperature higher than the temperature where the compound of Formula 2 can be maintained in a gas phase before reacting with AgOTf. For example, if n is an integer from 2 to 6, the AgOTf column may be heated to about 150° C. to about 250° C.

In another embodiment of the present disclosure, the un-reacted compound and other impurities may be removed by passing the [¹⁸F] fluoroalkyl compound through a filter before it is introduced into the heated AgOTf column. Any suitable filter known to adsorb un-reacted compounds or impurities can be used for the present disclosure. For example, silica gel Sep-Pak Cartridge (commercially obtainable from Waters, Inc.) can be used.

The process of preparing the [¹⁸F] fluoroalkylated radioactive compound by reacting [¹⁸F] fluoroalkyl triflate and a radioactive compound precursor is described. The radioactive compound precursor of the present disclosure has at least one functional group that can be [¹⁸F] fluoroalkylated by the [¹⁸F] fluoroalkyl triflate. The functional group(s) may independently be NH, OH or SH.

A person skilled in the art could select and use without any special difficulty such a compound having a functional group to be [¹⁸F] fluoroalkylated by [¹⁸F] fluoroalkyl triflate among known compounds. Some non-limiting examples of the radioactive compound precursor that can be used in the process according to the present disclosure are as follows:

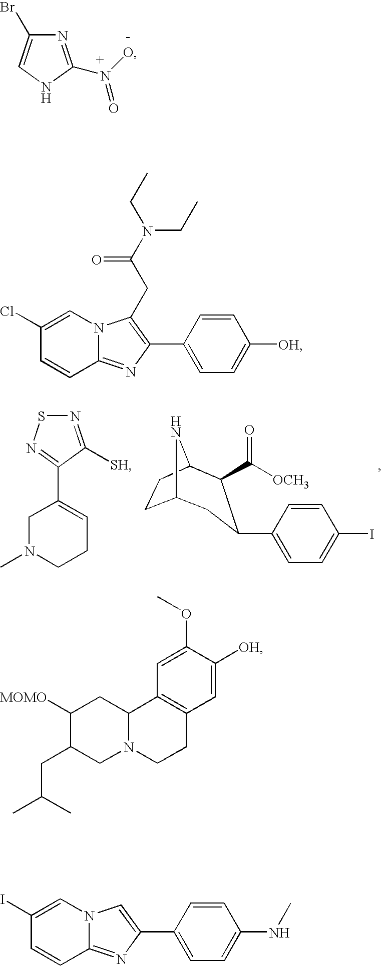

The above compounds may be commercially obtainable from ABX GmbH or can be synthesized using known processes. The radioactive compounds obtained by [¹⁸F] fluoroalkylating the above compounds can be used for PET.

Other than the radioactive compounds obtained by [¹⁸F] fluoroalkylating the above exemplary compounds, some non-limiting examples of radioactive compounds for PET that can be produced according to the present disclosure are as follows:

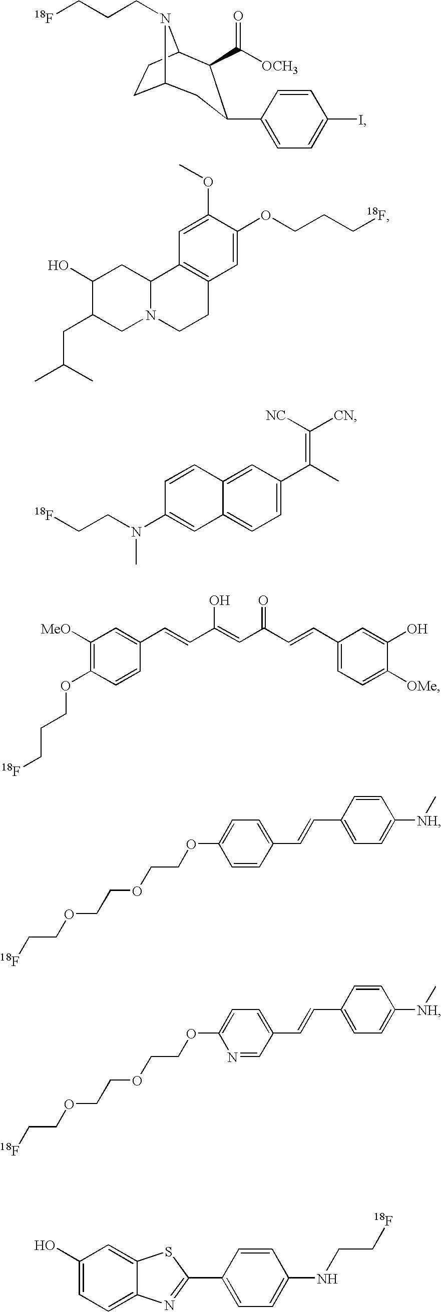

It will be appreciated that the above depicted compounds are only being disclosed to illustrate the radioactive compound precursors or the radioactive compounds of the present disclosure and are not meant to limit the scope of the preparation process according to the present disclosure in any way.

Since the [¹⁸F] fluoroalkyl triflate of the present disclosure has a highly reactive leaving group, the compound can be easily linked to the nitrogen, oxygen or sulfur atom of the radioactive compound precursor. Thus, the [¹⁸F] fluoroalkyl triflate generally reacts with the radioactive compound precursor within a short time at room temperature even without an alkaline agent. For example, a reaction vessel containing the radioactive compound precursor is placed at one end of the AgOTf column. Here, the vessel may be immersed in cold water so that the gas phase of [¹⁸F] fluoroalkyl triflate is captured in the liquid phase. As a result, a radioactive compound is formed from the reaction between the radioactive compound precursor and the [¹⁸F] fluoroalkyl triflate in the vessel.

If necessary, a desirable final product may be obtained by carrying out an additional reaction with respect to the radioactive compound produced according to the process of the present disclosure.

If necessary, a highly pure compound having radioactivity may be obtained by isolating and purifying the radioactive compound or the final product produced by the process of the present disclosure. Suitable methods known in the art including HPLC can be applied with respect to the isolation and purification.

If necessary, the radioactive compound or the final product obtained by the process of the present disclosure may be formulated to an injection solution which can be administered to a human or animal and may be applied to a disease diagnosis technique such as PET.

According to the process of the present disclosure, it is possible to obtain a radioactive compound in a high radiochemical yield. For example, a radiochemical yield of about 80% at maximum may be achieved when the compound of Formula 1 is transformed to the compound of Formula 2 using known methods. In forming the compound of Formula 3 by reacting the compound of Formula 2 with AgOTf, there is a high tendency of the halogen atom (Cl⁻, Br⁻ or I⁻) to bind to Ag⁺, resulting in a high radiochemical yield of the compound of Formula 3. In particular, the reaction using the heated AgOTf column has an almost 100% radiochemical yield. When the compound of Formula 3 is reacted with a radioactive compound precursor having a NH, OH or SH group, it is possible to achieve at least an about 95% radiochemical yield since TfO is a leaving group with a high leaving tendency. Therefore, in the consecutive process of starting from the compound of Formula 1, forming the compound of Formula 2, forming the compound of Formula 3 and then producing the radioactive compound by reacting the compound of Formula 3 with the radioactive compound precursor, an approximately 70% total radiochemical yield is expected even considering some loss when calculating the total yield. This is greater than the highest radiochemical yield (−50%) expected from conventional processes described in Reference 6.

Since the process according to the present disclosure corresponds to a two-step synthetic process, a higher radiochemical yield can be achieved compared to a one-step synthetic process.

The process according to the present disclosure involves a relatively short synthesis time even though it is a two-step synthetic process. One reason is because the compound of Formula 2 is transformed to the compound of Formula 3 very rapidly. Further, the isolation and purification processes for the produced radioactive compound are quite simple. Isolation and purification of the radioactive compound were difficult in conventional synthetic processes since they involve high temperature and the use of an alkaline agent and are thus likely to produce a significant amount of by-product. However, it is possible to easily isolate and purify the final product using the process according to the present disclosure, since the process uses only precursor compounds and organic solvents and can be carried out at room temperature. Thus, the process of the present disclosure involves reaction time as short as that of a one-step synthetic process.

In view of the above, by using the process according to the present disclosure, one can produce a large amount of radioactive compound of high quality within a short period of time.

### EXAMPLES

The following examples are provided for illustration of some of the various embodiments of the present disclosure but are by no means intended to limit the claimed scope.

### Synthesis of 3-Bromopropyl 1-(4-methylbenzene)sulfonate

To a solution of 3-bromo-1-propanol (1 g, 7.195 mmol) in pyridine (5 ml) was added dropwise TsCl (1.646 g, 8.634 mmol) at 0° C. The solution was stirred for 2 hours at room temperature. After the reaction was completed, ether (5 ml) was added to quench the reaction at 0° C. Then, the reaction mixture was extracted with water. The combined organic layers were dried over MgSO₄, filtered, and evaporated under reduced pressure. The crude product purified by silica gel column chromatography (hexane:EtOAc=4:1) provided 3-bromopropyl 1-(4-ethylbenzene)sulfonate (1.8 g, 85%) as a colorless oil.

### Obtaining Fluorine-18

An aqueous solution containing fluorine-18 isotopes was produced using a cyclotron. The solution was passed through a QMA light cartridge (commercially obtainable from Waters, Inc.) to adsorb fluorine-18 and then the adsorbed fluorine-18 was eluted with an acetonitrile solution (comprising 200 μL of water) in which Kryptofix 2.2.2. (K₂₂₂; commercially obtainable from Sigma Aldrich, corp.) 5 mg and KHCO₃ 0.73 mg were dissolved. The eluted isotope solution was heated to 100° C. under Ag gas in a glass vessel to evaporate moisture and organic solvents. 100 to 300 μL of the acetonitrile solution was additionally added 2 or 3 times, where moisture and organic solvents were all evaporated.

### Synthesis of 1-bromo-3-[¹⁸F] fluoropropane

To the dried mixture obtained in the above process, 200 μL of acetonitrile in which 30 μL of 3-bromopropyl 1-(4-methylbenzene)sulfonate was dissolved was added, and the mixture was heated to 120° C. for 20 minutes to synthesize 1-bromo-3-[¹⁸F] fluoropropane. The product was identified by TLC device for radioactivity measurement.

### Synthesis of 1-[¹⁸F] fluoro-3-triflate

Argon gas was introduced into the reaction container where 1-bromo-3-[¹⁸F] fluoropropane was synthesized and heated to 140° C. The vaporized compound was passed through an AgOTf column heated to 200° C. The flow rate of the argon gas was 10˜30 ml/min.

### Synthesis of [¹⁸F]FP-CIT

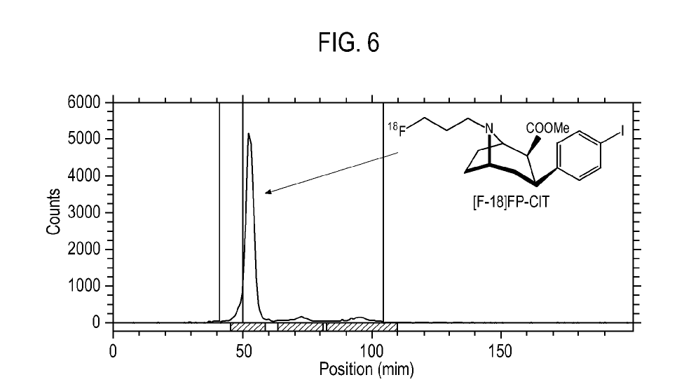

A reaction vessel containing 0.1 mg of nor-β-CIT (commercially obtainable from ABX GmbH) in 50 μL of 2-butanone was placed at one end of the AgOTf column, and the vessel was immersed into cold water so that 1-[¹⁸F] fluoro-3-triflate was captured in the solution. Afterwards, 2 mL of acetonitrile/ammonium formate buffer (50 mM) (50/50) was added to the reaction vessel for dilution, and [¹⁸F] FP-CIT was isolated using prep-HPLC. The isolation conditions were acetonitrile/Et₃N/H₂O (57.5/0.2/42.5), 4 ml/min and UV 254 nm, and the elution time was 44 to 48 minutes. Data for the synthesized [¹⁸F] FP-CIT, which was obtained using a TLC device for radioactivity measurement right before the isolation and purification process, is shown in FIG. 6.

Although the present disclosure has been described in detail with reference to certain embodiments thereof, other embodiments are possible within the spirit of the present disclosure. For example, it is possible to synthesize various structures of radioactive compounds by selecting various types of [¹⁸F] fluoroalkyl compound precursor and/or those of radioactive compound precursor.

What is claimed is:

1. A process of preparing a radioactive compound comprising:
forming a compound of Formula 3
[¹⁸F]F—CₙH₂ₙ—OTf (3)
by reacting a compound of Formula 2
[¹⁸F]F—CₙH₂ₙ—X (2)
wherein n is an integer from 2 to 6, and X is any one of Cl, Br and I,
with AgOTf; and
forming a radioactive compound containing fluorine-18 isotope by reacting the compound of Formula 3 with a radioactive compound precursor having at least one group selected from the group consisting of NH, OH and SH.

2. The process of claim 1, wherein AgOTf is present in a heated AgOTf column.

3. The process of claim 2, wherein the AgOTf column is heated to a temperature from about 150° C. to about 250° C.

4. The process of claim 2, wherein the compound of Formula 2 exists in a gas phase in the heated AgOTf column.

5. The process of claim 1, further comprising:
heating the compound of Formula 2 to at least a boiling point of the compound.

6. The process of claim 1, further comprising:
passing the compound of Formula 2 through a filter.

7. The process of claim 5, wherein n is 2, 3 or 4.

8. The process of claim 1, wherein the compound of Formula 2 is formed by a process comprising subjecting a compound of Formula 1
X′—CₙH₂ₙ—X (1)
wherein X′ is any one selected from the group consisting of TsO, NsO, MsO, TfO, BsO, Cl, Br and I,
to substitution with a fluorine-18 isotope.

9. The process of claim 8, further comprising:
heating the compound of Formula 2 to at least a boiling point of the compound.

10. A process of preparing a radioactive compound comprising:
forming a compound of Formula 2
[¹⁸F]F—CₙH₂ₙ—X (2)
by subjecting a compound of Formula 1
X′—CₙH₂ₙ—X (1)
wherein n is a integer from 2 to 6, X′ is any one selected from the group consisting of TsO, NsO, MsO, TfO, BsO, Cl, Br and I, and X is any one of Cl, Br and I,
to substitution with a fluorine-18 isotope;
heating the compound of Formula 2 to at least a boiling point of the compound;
forming a compound of Formula 3
[¹⁸F]F—CₙH₂ₙ—OTf (3)
by reacting the compound of Formula 2 with AgOTf; and
forming a radioactive compound containing fluorine-18 isotope by reacting the compound of Formula 3 with a radioactive compound precursor having at least one group selected from the group consisting of NH, OH and SH.

11. The process of claim 10, wherein AgOTf is present in a heated AgOTf column.

12. The process of claim 11, wherein the AgOTf column is heated to a temperature from about 150° C. to about 250° C.

13. The process of claim 11, wherein the compound of Formula 2 exists in a gas phase in the heated AgOTf column.

14. The process of claim 10, wherein n is 2, 3 or 4.

15. The process of claim 10, further comprising:
passing the compound of Formula 2 through a filter and then reacting with AgOTf.

16. The process of claim 1, wherein the radioactive compound precursor is represented by any one of the following chemical structures:

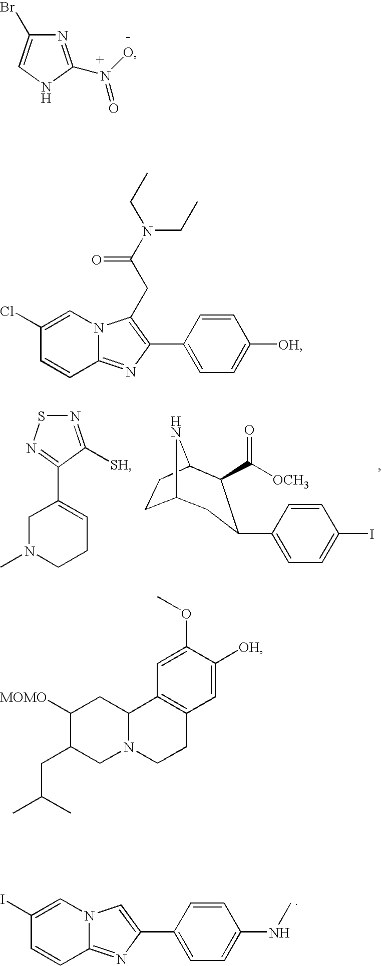

17. The process of claim 2, wherein the radioactive compound precursor is represented by any one of the following chemical structures:

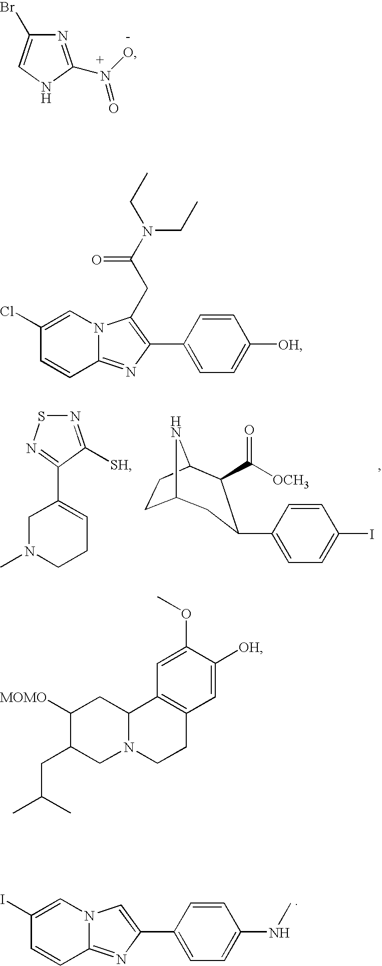

18. The process of claim 3, wherein the radioactive compound precursor is represented by any one of the following chemical structures:

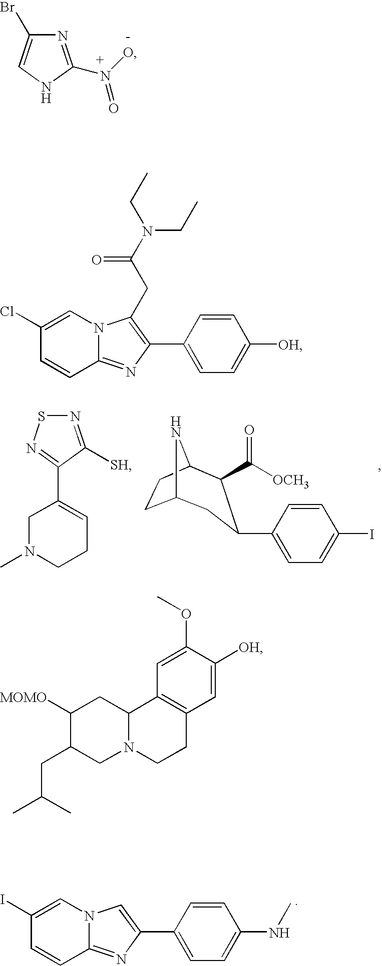

19. The process of claim 10, wherein the radioactive compound precursor is represented by any one of the following chemical structures:

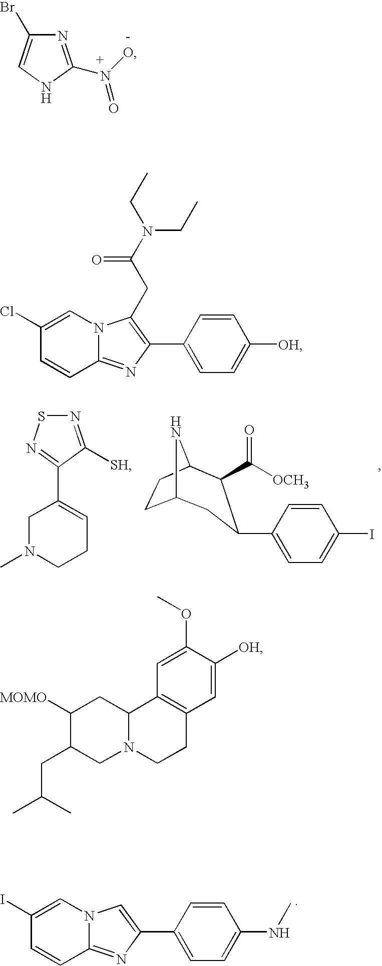

20. The process of claim 11, wherein the radioactive compound precursor is represented by any one of the following chemical structures:

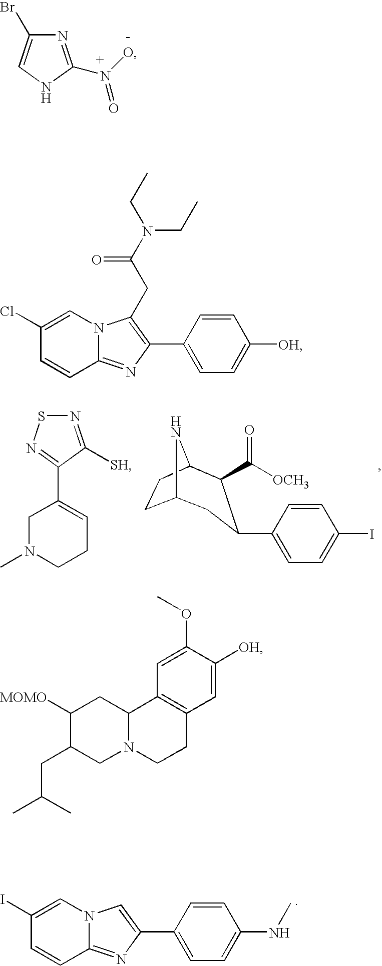

21. The process of claim 12, wherein the radioactive compound precursor is represented by any one of the following chemical structures:

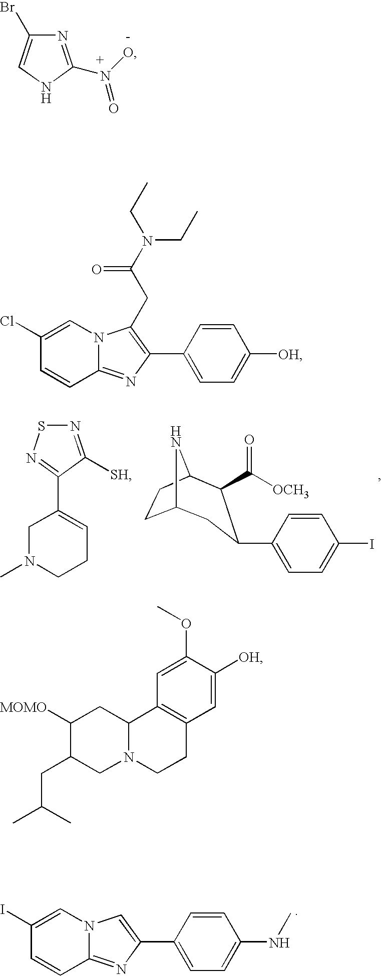

22. The process of claim 1, wherein the radioactive compound is used for positron emission tomography (PET).

23. The process of claim 22, wherein the radioactive compound is represented by any one of the following chemical structures:

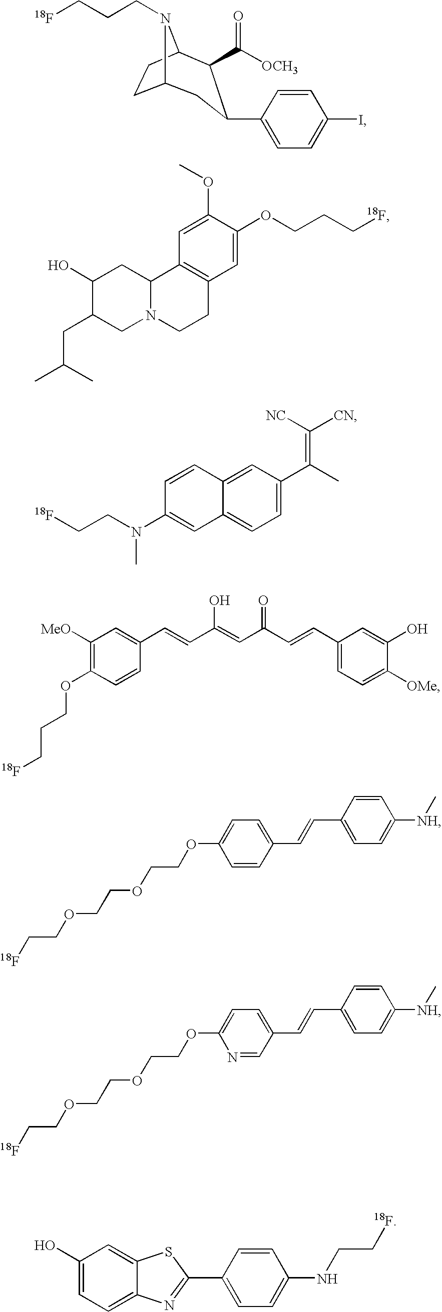

24. The process of claim 2, wherein the radioactive compound is used for positron emission tomography (PET).

25. The process of claim 3, wherein the radioactive compound is used for positron emission tomography (PET).

26. The process of claim 10, wherein the radioactive compound is used for positron emission tomography (PET).

27. The process of claim 11, wherein the radioactive compound is used for positron emission tomography (PET).

28. The process of claim 12, wherein the radioactive compound is used for positron emission tomography (PET).
